(19) United States
(12) Patent Application Publication
(10) Pub. No.: US 2016/0108005 A1
(43) Pub. Date: Apr. 21, 2016

(54) TREATMENT OR PREVENTION OF DEPRESSION USING MENTHOL AND/OR ICILIN

(71) Applicant: NESTEC S.A., Vevey (CH)

(72) Inventors: Susana CAMACHO, Lausanne (CH); Stephanie MICHLIG-GONZALEZ, Le Mont-sur-Lausanne (CH); Johannes LE COUTRE, Pully (CH); Henry MARKRAM, Lausanne (CH); Maurizio PEZZOLI, Lausanne (CH)

(21) Appl. No.: 14/892,016

(22) PCT Filed: May 23, 2014
(86) PCT No.: PCT/EP2014/060634

§ 371 Date: Nov. 18, 2015

Related U.S. Application Data
(60) Provisional application No. 61/827,256, filed on May 24, 2013.

(51) Int. Cl. C07D 239/36; C07C 35/12

(52) U.S. Cl. CPC C07D 239/36; C07C 35/12; A23L 1/30; A23V 2002/00

(57) ABSTRACT

Compositions for treatment or prevention of depression are provided, and the compositions contain a therapeutically effective amount of a compound selected from the group consisting of Menthol, Icilin and combinations thereof. Methods for treatment or prevention of depression are also provided, and the methods include administering such compositions.

### BACKGROUND

The present disclosure generally relates to methods and compositions for prevention or treatment of depression. More specifically, the present disclosure relates to compositions comprising at least one of Menthol or Icilin and further relates to methods comprising administering such compositions.

Severe depression is a long lasting and recurring disease, which is usually poorly diagnosed. Furthermore, many patients suffer from mild or moderately severe depression. Conventional anti-depressants still have many limitations that hinder the effective treatment of depression. The glutamatergic system may be a potential target for anti-depressant therapy. Glutamate antagonists inhibit the binding of glutamate to NMDA receptors such that accumulation of Ca²⁺ and therefore excitotoxicity can be avoided. However, use of glutamate antagonists presents a huge obstacle because the treatment interferes with the normal action of glutamate under standard conditions.

There is a need for compounds for the treatment or prevention of mental diseases and/or disorders which do not show the negative side effects of known anti-depressants. Many patients are interested in alternative therapies which could minimize the side effects associated with high doses of drugs and/or yield additive clinical benefits. Thus, there is an increasing interest in the development of compounds as well as pharmaceutical and/or dietary compositions that may be used to treat mental diseases/disorders, prevent the development of mental diseases/disorders such as depression, and stabilize mood.

### SUMMARY

The present inventors surprisingly and unexpectedly found that an active compound from spices, the transient receptor potential M8 (TRPM8) channel agonist Menthol, can depress neuronal activity in the neocortex and the amygdale. The present inventors discovered the same effect with Icilin, a synthetic super-agonist of the TRPM8 ion channel, even though the structure of Icilin is not related to Menthol.

Accordingly, in a general embodiment, the present disclosure provides a method for treating depression. The method comprises administering to an individual having depression a therapeutically effective amount of a compound selected from the group consisting of Menthol, Icilin and combinations thereof.

In a related embodiment, the depression is selected from the group consisting of unipolar depression, bipolar depression, acute depression, chronic depression, sub-chronic depression, dysthymia, postpartum depression, climacteric depressive symptoms, seasonal affective disorders, and combinations thereof.

In a related embodiment, the composition is administered periodically for at least one year. The composition can be administered daily.

In a related embodiment, the composition is selected from the group consisting of a medicament, a food product, and a supplement to a food product.

In another embodiment, a method for preventing depression is provided. The method comprises administering to an individual at risk of same a therapeutically effective amount of a compound selected from the group consisting of menthol, icilin and combinations thereof.

In a related embodiment, the depression is selected from the group consisting of unipolar depression, bipolar depression, acute depression, chronic depression, sub-chronic depression, dysthymia, postpartum depression, climacteric depressive symptoms, seasonal affective disorders, and combinations thereof.

In a related embodiment, the composition is administered periodically for at least one year. The composition can be administered daily.

In a related embodiment, the composition is selected from the group consisting of a medicament, a food product, and a supplement to a food product.

In another embodiment, a composition for treating or preventing depression is provided. The food product comprises a therapeutically effective amount of a compound selected from the group consisting of Menthol, Icilin and combinations thereof.

In a related embodiment, the composition is the composition is a medicament.

In a related embodiment, the composition is a food product. The food product can comprise a component selected from the group consisting of protein, carbohydrate, fat and combinations thereof.

In a related embodiment, the composition is a supplement to a food product.

An advantage of the present disclosure is to prevent or treat depression more effectively and/or more safely than glutamate antagonists.

Another advantage of the present disclosure is to prevent or treat depression without interfering with the normal action of glutamate under standard conditions.

Still another advantage of the present disclosure is to prevent or treat depression with compounds that can be easily and safely used in food products.

Yet another advantage of the present disclosure is to prevent or treat depression by targeting the pre-synaptic phase of neuronal firing.

An additional advantage of the present disclosure is to prevent or treat depression by targeting the pre-synaptic phase of neuronal firing while reducing the possibility of excitotoxicity.

Another advantage of the present disclosure is to prevent or treat depression with naturally-occurring compounds that can be found in spices.

Still another advantage of the present disclosure is to prevent or treat depression with tolerable side effects or no side effects.

Additional features and advantages are described herein, and will be apparent from, the following Detailed Description and the Figures.

### BRIEF DESCRIPTION OF THE FIGURES

FIG. 1 shows the chemical structures of compounds that can be used in embodiments of the composition according to the present disclosure.

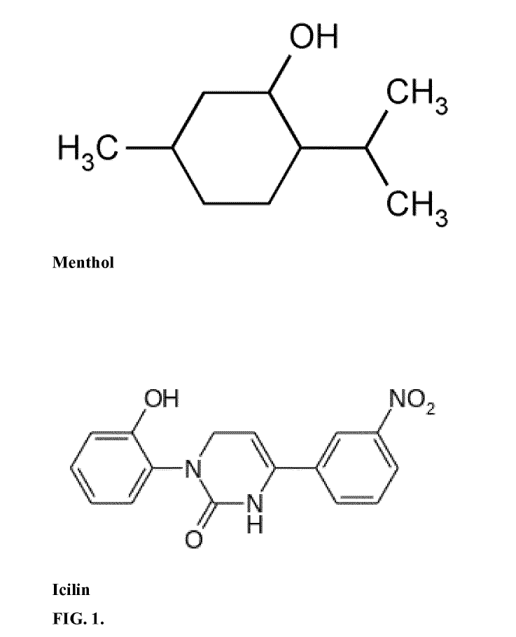

FIG. 2 shows charts of whole cell, current clamp recordings in a Lateral Amygdala glutamatergic neuron (in a mouse brain slice) in the absence (control) and presence of TRPM8 ligands (Linalool, Icilin or Menthol).

FIG. 3 shows a chart of whole cell, current clamp recordings in a Lateral Amygdala glutamatergic neuron (in a mouse brain slice) with increasing concentration of gabazine (GABA A blocker) applied extracellularly during recordings of 5 min each (washout 10 min).

FIG. 4 shows a chart of whole cell, current clamp recordings in a Lateral Amygdala glutamatergic neuron (in a mouse brain slice) showing enhanced detail of a burst.

FIG. 5 shows a chart of whole cell, current clamp recordings in a Lateral Amygdala glutamatergic neuron (in a mouse brain slice) with increasing concentration of gabazine (GABA A blocker) applied extracellularly during recordings of 5 min each (washout 10 min) while 10 minutes previous to and during the exposure of the different concentrations of gabazine, 250 μM menthol was also applied extracellularly.

### DETAILED DESCRIPTION

All percentages expressed herein are by weight of the total weight of the composition unless expressed otherwise. When reference is made to the pH, values correspond to pH measured at 25° C. with standard equipment. As used in this disclosure and the appended claims, the singular forms “a,” “an” and “the” include plural referents unless the context clearly dictates otherwise. As used herein, “about” is understood to refer to numbers in a range of numerals. Moreover, all numerical ranges herein should be understood to include all integers, whole or fractions, within the range. The food composition disclosed herein may lack any element that is not specifically disclosed herein. Thus, “comprising” includes “consisting essentially of” and “consisting of.”

As used herein, “depression” includes, as non-limiting examples, unipolar depression, bipolar depression, acute depression, chronic depression, sub-chronic depression, dysthymia, postpartum depression, climacteric depressive symptoms, and seasonal affective disorders.

“Prevention” includes reduction of risk and/or severity of depression. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

As used herein, a “therapeutically effective amount” is an amount that prevents a deficiency, treats a disease or medical condition in an individual or, more generally, reduces symptoms, manages progression of the diseases or provides a nutritional, physiological, or medical benefit to the individual.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human, having or at risk for a medical condition that can benefit from the treatment.

“Food product” and “food composition,” as used herein, are understood to include any number of optional additional ingredients, including conventional food additives, for example one or more of proteins, carbohydrates, fats, acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers and/or vitamins. The optional ingredients can be added in any suitable amount.

As set forth above, the present inventors surprisingly and unexpectedly found that an active compound from spices, the transient receptor potential M8 (TRPM8) channel agonist Menthol, can depress neuronal activity in neocortex and amygdale. The present inventors discovered the same effect with Icilin, a synthetic super-agonist of the TRPM8 ion channel, even though the structure of Icilin is not related with Menthol; nevertheless, Icilin produces an extreme sensation of cold both in humans and animals. These natural compounds reduce neuronal excitability by 1) increasing the threshold to trigger an action potential and consequently increasing the amount of current required to trigger an action potential in the neocortex; and 2) abortion of action potentials at higher stimulation levels, most likely related to the use-dependent block of Na⁺ channels in the neocortex and lateral amygdale. These active compounds change the firing patterns especially at higher stimulation levels where a progressive and dramatic reduction of the action potential (APs) amplitude occurs until complete abortion of APs.

Without wishing to be bound by theory, the inventors believe that the mechanism underlying the selected active compounds of spices, namely Menthol and Icilin, solves two main problems compared to neuroprotective glutamate antagonists: 1) Menthol and Icilin target a presynaptic phase of APs, decreasing activity and diminishing glutamate release, which reduces drastically the possibility of reaching excitotoxicity levels; and 2) Menthol and Icilin act stronger in the high stimulation context. In contrast to glutamate antagonists that typically inhibit the binding of glutamate to NMDA receptors, Menthol and Icilin decrease neuronal activity, and target the pre-synaptic phase of the firing to reduce the possibilities of excitotoxicity one step earlier.

Accordingly, the composition provided by the present disclosure comprises a therapeutically effective amount of at least one of Menthol or Icilin. In an embodiment, depression is treated or prevented by administering to an individual in need of same the composition comprising at least one of Menthol or Icilin. For example, the composition comprising at least one of Menthol or Icilin can be administered to an individual having depression to treat the depression. The depression can be unipolar depression, bipolar depression, acute depression, chronic depression, sub-chronic depression, dysthymia, postpartum depression, climacteric depressive symptoms, seasonal affective disorders, and combinations thereof. In an embodiment, the composition can be administered periodically for at least one year, preferably at least two years, more preferably at least three years, even more preferably at least four years, and most preferably at least five years.

Each of Menthol and/or Icilin can be administered to the individual in a daily amount of 0.0015 mg/kg of body weight to 400 mg/kg of body weight, preferably 0.1 mg/kg of body weight to 300 mg/kg of body weight, more preferably 1.0 mg/kg of body weight to 200 mg/kg of body weight, and most preferably 10.0 mg/kg of body weight to 100 mg/kg of body weight. For example, each of Menthol and/or Icilin can be administered to the individual in a daily amount of 0.0015 mg/kg of body weight to 0.01 mg/kg of body weight, 0.01 mg/kg of body weight to 0.1 mg/kg of body weight, 0.1 mg/kg of body weight to 1.0 mg/kg of body weight, 1.0 mg/kg of body weight to 10.0 mg/kg of body weight, 10.0 mg/kg of body weight to 100.0 mg/kg of body weight, 100.0 mg/kg of body weight to 200.0 mg/kg of body weight, 200.0 mg/kg of body weight to 300.0 mg/kg of body weight, or 300.0 mg/kg of body weight to 400.0 mg/kg of body weight.

The composition comprising at least one of Menthol or Icilin may be a medicament, a food product or a supplement to a food product. The supplement may be in the form of tablets, capsules, pastilles or a liquid, for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

The supplement can be added in a product acceptable to the consumer as an ingestible carrier or support. Non-limiting examples of such carriers or supports are a pharmaceutical, a food composition, and a pet food composition. Non-limiting examples for food and pet food compositions are milks, yogurts, curds, cheeses, fermented milks, milk-based fermented products, fermented cereal based products, milk-based powders, human milks, preterm formulas, infant formulas, oral supplements, and tube feedings.

In an embodiment, the composition comprising at least one of Menthol or Icilin is administered to a human. In an alternative embodiment, the composition comprising at least one of Menthol or Icilin is administered to a non-human animal, preferably a cat or a dog. Advantageously, the composition can be provided to a companion animal by its owner.

### EXAMPLES

The following non-limiting examples present scientific data developing and supporting the concept of treatment or prevention of neurodegenerative disorders using Menthol and Icilin.

A mouse brain slice was used to study the effects of Menthol, Linalool (another transient receptor potential M8 (TRPM8) channel agonist) and Icilin. The amygdaloid complex is located within the medial temporal lobe in neocortex and amygdala. The lateral and basolateral nuclei of the amygdaloid complex receive sensory information from cortical and thalamic structures, process the information, and then transmit the information, either directly or through the basal nucleus, to the central nucleus. For experimental analysis of neuronal activity, synaptic responses from the basolateral complex can be evoked electrically using electrodes, and the action potentials can be measured.

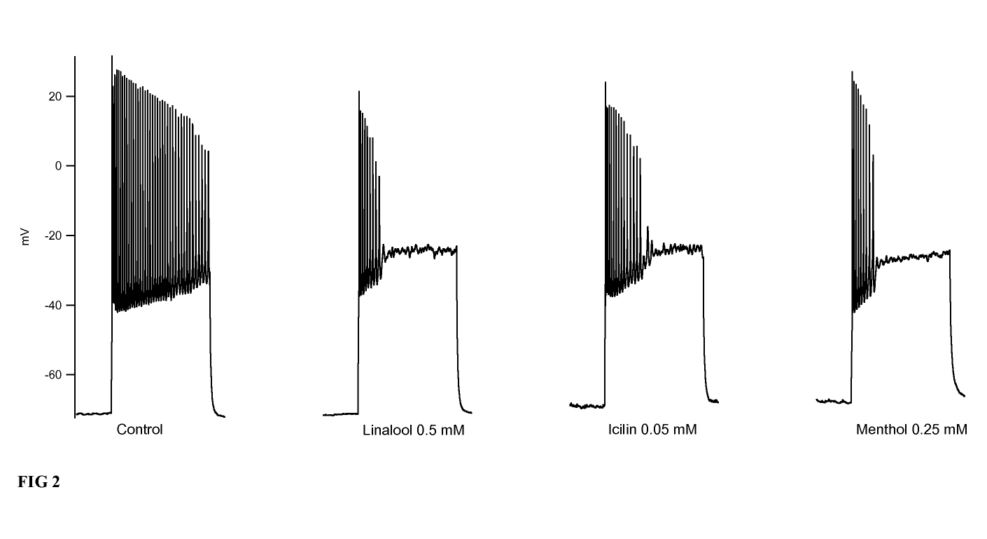

FIG. 2 shows recordings in the absence of Menthol, Linalool or Icilin (control) and recordings in the presence of Menthol, Linalool or Icilin. A square pulse of 2.5 s was applied at high depolarization of membrane potential (approximately −30 mV). The recordings show that, in the presence of the TRPM8 ligands at high depolarization levels, inactivation of the sodium fast channels happens sooner relative to control, avoiding further neuronal firing.

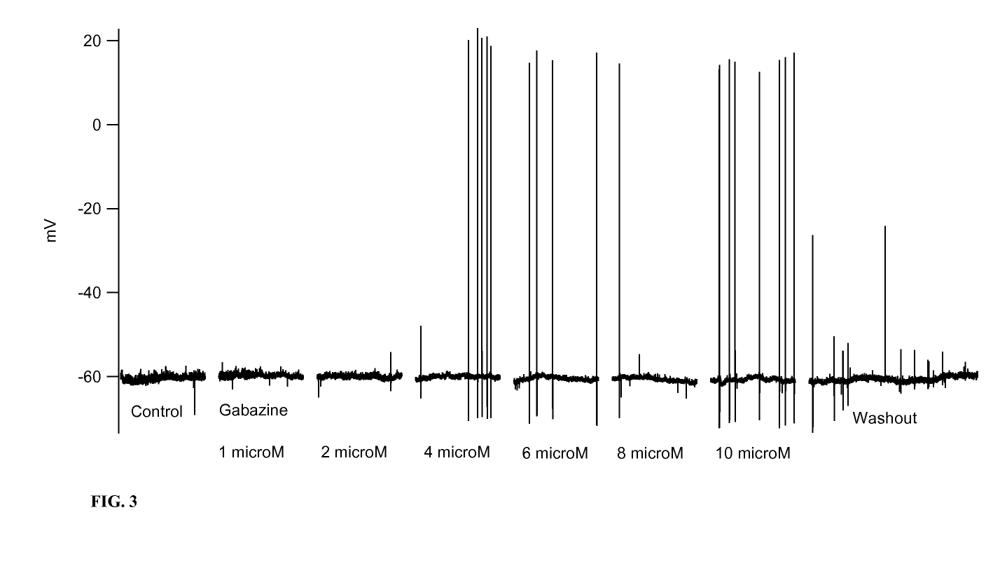

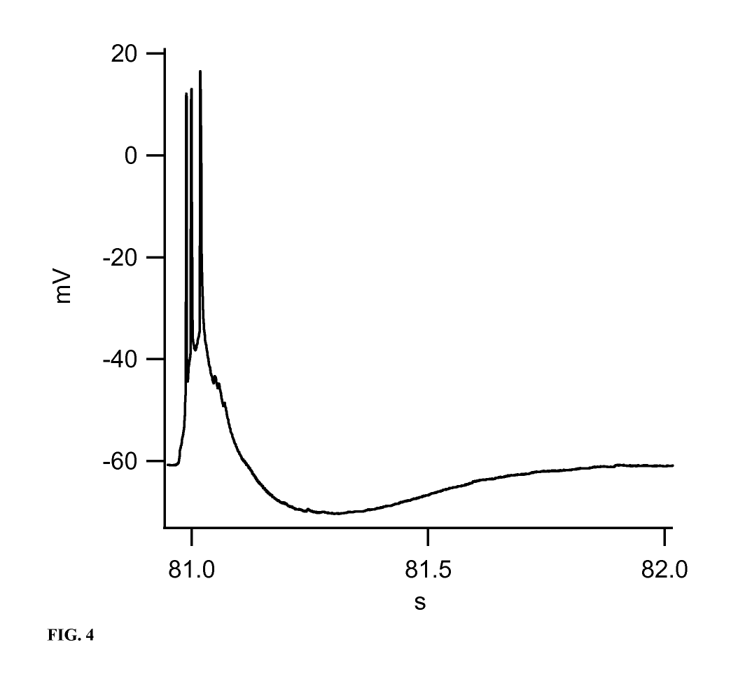

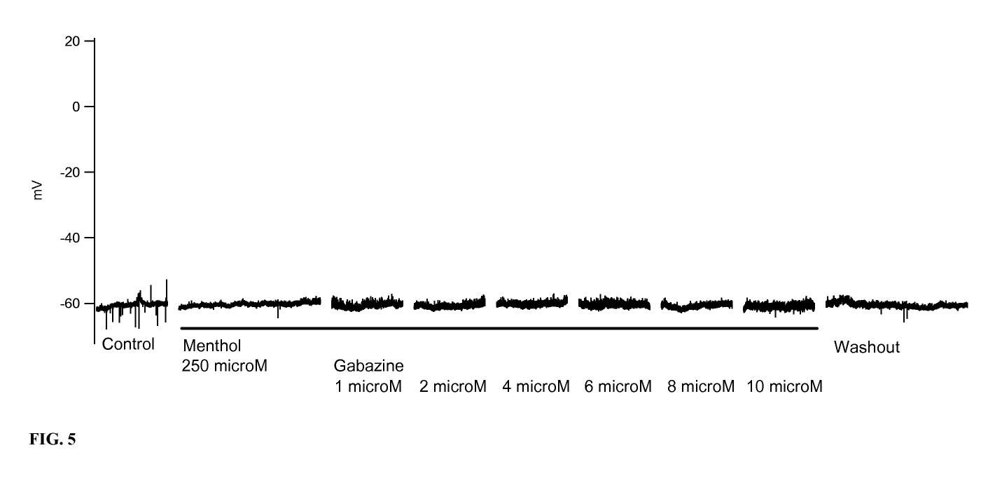

FIG. 3 shows recordings in increasing concentrations of gabazine, a GABA A blocker, applied extracellularly during recordings of 5 minutes each with 10 minute washout. As shown, neurons spontaneously present action potential bursts due to massive presynaptic discharges. FIG. 4 depicts enhanced detail of one of the bursts and shows that serial action potentials can be observed in a single burst. For comparison to FIG. 3, FIG. 5 shows recordings under the same conditions, namely increasing concentrations of gabazine applied extracellularly during recordings of 5 minutes each with 10 minute washout, except that in FIG. 5, Menthol 250 μM was applied extracellularly at 10 minutes previous to and during the exposure of the different concentrations of gabazine. As illustrated in the figure, neurons show a complete absence or a strongly decreased presence of spontaneous bursts (compare FIG. 5 to FIG. 3).

These experimental results demonstrate that Menthol and Icilin increase the threshold to trigger an action potential and consequently increase the amount of current required to trigger an action potential in the neocortex, and also abort action potentials at higher stimulation levels.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treating depression comprising administering to an individual having depression a therapeutically effective amount of a compound selected from the group consisting of Menthol, Icilin and combinations thereof.

2. The method of claim 1 wherein the depression is selected from the group consisting of unipolar depression, bipolar depression, acute depression, chronic depression, sub-chronic depression, dysthymia, postpartum depression, climacteric depressive symptoms, seasonal affective disorders, and combinations thereof.

3. The method of claim 1 wherein the composition is administered periodically for at least one year.

4. The method of claim 1 wherein the composition is administered daily.

5. The method of claim 1 wherein the composition is selected from the group consisting of a medicament, a food product, and a supplement to a food product.

6. A method for preventing depression comprising administering to an individual at risk of same a therapeutically effective amount of a compound selected from the group consisting of Menthol, Icilin and combinations thereof.

7. The method of claim 6 wherein the depression is selected from the group consisting of unipolar depression, bipolar depression, acute depression, chronic depression, sub-chronic depression, dysthymia, postpartum depression, climacteric depressive symptoms, seasonal affective disorders, and combinations thereof.

8. The method of claim 6 wherein the composition is administered periodically for at least one year.

9. The method of claim 6 wherein the composition is administered daily.

10. The method of claim 6 wherein the composition is selected from the group consisting of a medicament, a food product, and a supplement to a food product.

11. A composition for treating or preventing depression comprising a therapeutically effective amount of Menthol and Icilin.

12. The composition of claim 11 wherein the composition is a medicament.

13. The composition of claim 11 wherein the composition is a food product.

14-15. (canceled)
